(19) United States
(12) Patent Application Publication
(10) Pub. No.: US 2013/0017201 A1
(43) Pub. Date: Jan. 17, 2013

(54) BISPECIFIC MONOCLONAL ANTIBODY CAPABLE OF CROSS REACTING WITH LETHAL FACTOR (LF) AND EDEMA FACTOR (EF), AND NEUTRALIZING EDEMA TOXIN (ET) AS WELL AS LETHAL TOXIN (LT) OF BACILLUS ANTHRACIS

(71) Applicants: Rakesh Bhatnagar, New Delhi (IN); Parul Kulshreshtha, New Delhi (IN)

(72) Inventors: Rakesh Bhatnagar, New Delhi (IN); Parul Kulshreshtha, New Delhi (IN)

(73) Assignee: Rakesh BHATNAGAR, New Delhi (IN)

(21) Appl. No.: 13/636,239

(22) PCT Filed: Mar. 22, 2011
(86) PCT No.: PCT/IN2011/000191

§ 371 Date: Sep. 20, 2012

(30) Foreign Application Priority Data
Mar. 25, 2010 (IN) 714/DEL/2010

(51) Int. Cl. C07K 16/46; C12N 9/96; A61P 31/04; C12P 21/08; A61K 39/395

(52) U.S. Cl. 424/136.1; 530/387.3; 435/70.21; 435/188; 530/391.3

(57) ABSTRACT

The present invention relates to a monoclonal antibody (mAb) having capabilities of binding with lethal factor (LF) as well as edema factor (EF), and neutralizing lethal toxin (LT) as well as edema toxin (ET) of B. anthracis. It also relates to process for preparation of said mAb, and to pharmaceutical preparations, anthrax diagnostic tool, in-vivo diagnostic imaging tool comprising said mAb. It also relates to genetically modified mAb, and method for prophylaxis against anthrax disease comprising administration of mAb or genetically modified mAb of present invention.

### FIELD OF THE INVENTION

The invention relates generally to the field of immunology, particularly to a bispecific monoclonal antibody capable of cross reacting with lethal factor (LF) as well as edema factor (EF), and neutralizing both toxins—lethal toxin [LT] as well as edema toxin [ET] of Bacillus anthracis.

### BACKGROUND OF THE INVENTION

Anthrax is a highly lethal infectious disease caused by the spore-forming bacterium Bacillus anthracis. The deliberate distribution of anthrax spores through US mail system in 2001 resulted in 5 deaths among the 11 individuals who contracted inhalational anthrax, which highlight the great threat posed by the potential use of anthrax in terrorism and warfare. The lethality of inhalational anthrax is primarily due to the action of anthrax toxins. Bacterium produces three toxin components, they are protective antigen (PA), lethal factor (LF), and edema factor (EF).

The PA together with LF forms lethal toxin (LT), and PA together with EF forms edema toxin (ET). The PA functions as a vehicle to mediate the cellular uptake of the LF and EF.

The LF is a zinc-dependent endopeptidase that cleaves mitogen-activated protein kinase kinases and can replicate symptoms of anthrax when injected in animals with PA.

The EF is a calcium-calmodulin-dependent adenylate cyclase with a range of toxic effects in the host.

The toxins—LT and ET, respectively formed due to binding of PA with LF and EF are the dominant virulence factors for anthrax.

Currently there are no approved therapies for anthrax except antibiotics. The treatment with antibiotics has considerable limitations. Exposure to the bacterium followed by bacterial division leads to the production of large quantities of anthrax toxins. Thus, unless exposure is diagnosed early enough for vigorous antibiotic treatment, patients succumb to disease due to high systemic levels of lethal and edema toxins [LT and ET].

Presently, the immunization against anthrax is achieved by Anthrax Vaccine Adsorbed (AVA) vaccine, which is active immunization in nature. The AVA is alum adsorbed culture filtrate of the non-pathogenic strain of B. anthracis, containing Protective Antigen as its major immunogenic component. However, this methodology is not efficient at protecting newly infected individuals. Further, it requires repeated administration and at least 4 weeks for development of protective titers.

In order to counteract the limitations of antibiotics and active immunization, therapeutic strategies that evoke protection against anthrax by targeting either PA or LF have been tried. With aim to overcome drawbacks of antibiotics, the lethal factor (LF) and edema factor (EF) have been studied and found to play a role against anthrax, but in providing active immunity. The passive immunization using monoclonal antibodies from mammalian source may be representing an attractive alternative for prevention of anthrax.

An anti-LF neutralizing monoclonal antibody (mAb), cross-reactive with EF, has been studied, but it did not show anti-ET neutralizing function. Accordingly, even the anti-LF mAb has not been found to be effective to neutralize the effects of both toxins—LT and ET, and hence, has not been found to be effective to save the patients already infected with Anthrax.

Therefore, there is a need for anthrax therapy that not only cross reacts with (or recognizes) lethal factor (LF) and edema factor (EF), but also neutralizes the effects of their respective anthrax toxins [LT and ET], and hence, can achieve the passive immunization, so that the patients already infected with anthrax could be saved by use of therapy.

Recently, Protective Antigen (PA) has been a primary target for passive protection (WO2007/084107), therefore, currently available monoclonal anti-anthrax antibodies target Protective Antigen (PA).

However, it is believed that PA may be mutated within currently known monoclonal antibodies (mAb) neutralizing epitopes, therefore, anti-PA therapies are no longer effective.

Administration of anti-LF neutralizing mAb (WO2006/096039) has also been tried for passive immunization. However, such approach will only neutralize LT and not ET. Therefore, even this approach is not acceptable for complete cure of patients infected with Anthrax.

Co-administration of PA and LF specific monoclonal antibodies (WO 2007/123562) for protective therapy by combinatorial treatment with anti-PA and anti-LF antibodies has also been tried. However, such approach requires injection of large amounts of at least two monoclonal antibodies into the patient's body, which is expected to result in allergic response due to accumulation of large amounts of monoclonal antibodies in the body. Therefore, even this approach has not been adopted for treatment of anthrax.

Therefore, again, the research was diverted to find antibodies specifically suitable against individual toxins—LT or ET by binding and neutralizing LF or EF that could help in curbing pathogenesis. Recently, in WO2008/103845, certain monoclonal antibodies and their modified versions (engineered antibodies), F(ab′)₂, Fab, Fv and Fd, have been reported. As per this patent application, the reported antibodies are capable of, separately, targeting either Lethal Factor (LF) or Edema Factor (EF).

Despite the ability to induce protective immunity with AVA or recombinant PA immunization, widespread immunization against anthrax may not be practical because of the heavy cost involved in immunizing the entire population. As the number of people actively infected after release of anthrax spores used as biological weapon may represent only a fraction of the entire population, the choice to have immunization of entire population is not justified. Therefore, the society looks for a therapy to cure anthrax by passive immunization, which due to the unpredictable nature of bio-terrorism and absence of real-time detection systems is now unavoidable for an efficient post-exposure therapy for Bacillus anthracis infection.

The symptoms of anthrax appear due to very high circulating toxins in the blood, therefore, the future strategies have to be designed to neutralize or at least to bring down the systemic toxin levels of both toxins—LT and ET. It has also been observed that a monoclonal antibody may be capable of binding to or cross react with LF or EF, but it is not necessary that it will also neutralize their respective toxin—LT/ET.

### Need of the Invention

Therefore, there is a need to have best solution for passive immunization against anthrax by providing an anti-anthrax therapy wherein a single monoclonal antibody which should not only be capable of cross reacting with (or recognizing) lethal factor (LF) as well as with edema factor (EF), but should also be capable of neutralizing the effects of their respective anthrax toxins [LT and ET], so that the patients already infected with anthrax could be saved by use of single monoclonal antibody, and hence, probability of allergic reactions is at least avoided.

### Objects of the Invention

Accordingly, main object of present invention is to provide solution for passive immunization of anthrax by providing an anthrax therapy wherein a single therapeutic monoclonal antibody is not only capable of cross reacting with (or binding or recognizing) lethal factor (LF) as well as edema factor (EF), but is also capable of neutralizing anthrax toxins—lethal toxin (LT) and edema toxin (ET) of B. anthracis, so that the patients already infected with anthrax could be saved by use of a single monoclonal antibody, and hence, probability of allergic reactions is at least avoided.

This is also an object of the present invention to provide a therapy which is capable of treating patients already infected with anthrax and who could not be diagnosed at early stages of infection.

This is also an object of present invention to provide a therapeutic monoclonal antibody which is capable of treating the patients already infected with B. anthraciseven if they were not vaccinated prior to the infection or could not be diagnosed at early stages of infection.

Another object of present invention is to provide a bispecific monoclonal antibody which is capable of combining with pharmaceutically acceptable carrier to result in a protective pharmaceutical preparation.

Yet another object of present invention is to provide a bispecific monoclonal antibody which is capable of being combined with an antibiotic regimen as a method for the treatment or amelioration of anthrax disease.

Still another object of present invention is to provide a bispecific monoclonal antibody which is capable of binding to different carriers and being used in in-vitro studies to detect the presence of LF and/or EF for the design of an anthrax diagnostic tool.

Another object of present invention is also to provide a bispecific monoclonal antibody which is capable of being labeled with a paramagnetic or a radioisotope for in-vivo diagnostic imaging to assess the progress of disease in anthrax patients.

This is also an object of present invention to provide a bispecific monoclonal antibody which is capable of protecting mice from lethal toxin challenge when it is pre-administered to mice.

This is also an object of present invention to provide a bispecific monoclonal antibody which is capable of protecting mice from edema toxin challenge when it is pre-administered to mice.

This is also an object of present invention to provide a bispecific monoclonal antibody which is capable of protecting mice from lethal anthrax challenge when it is administered 24 hours after challenge either alone or in combination with antibiotic to mice.

Other objects and advantages of present invention will be more apparent from the following description particularly when it is read in conjunction with accompanying figures which are not intended to limit scope of present invention.

### BRIEF DESCRIPTION OF ACCOMPANYING FIGURES

FIG. 1 illustrates capability of monoclonal antibody of present invention to recognize (to cross react with or bind with) Lethal Factor (LF) and Edema Factor (EF) as observed by Solid Phase Enzyme Linked Immunosorbent Assay (ELISA).

FIG. 2 illustrates capability of monoclonal antibody of present invention to recognize Lethal Factor (LF) and Edema Factor (EF) as observed by Western Blotting, wherein FIGS. 2a illustrates capability to recognize recombinant Edema Factor (EF) and FIG. 2b illustrates capability to recognize recombinant Lethal Factor (LF) by monoclonal antibody of present invention.

FIG. 3 illustrates capability of monoclonal antibody of present invention to neutralize Lethal Toxin (LT) on J774A.1 cell line in accordance with one of the embodiments of present invention.

FIG. 4 illustrates capability of monoclonal antibody of present invention to neutralize Edema Toxin (ET) on CHO.K1 cell line in accordance with one of the embodiments of present invention.

FIG. 5 illustrates capability of monoclonal antibody of present invention to protect animal from intraperitoneal anthrax challenge on its pre-administration in accordance with one of the embodiments of present invention.

FIG. 6 illustrates capability of monoclonal antibody of present invention to protect animal from intraperitoneal Lethal Toxin (LT) challenge in accordance with one of the embodiments of present invention.

FIG. 7 illustrates capability of monoclonal antibody of present invention to protect animal from intraperitoneal Edema Toxin (ET) challenge in accordance with one of the embodiments of present invention.

FIG. 8 illustrates capability of monoclonal antibody of present invention along with antibiotic dose to protect animal from intraperitoneal anthrax challenge in accordance with one of the embodiments of present invention.

### DESCRIPTION AND PREFERRED EMBODIMENTS OF THE INVENTION

### Abbreviations:

In the following description, the term PBS means Phosphate Buffered Saline; PBST means PBS supplemented with 0.1% of Tween 20; FBS means Fetal Bovine Serum; IgG means Immunoglobulin G; NBT means Nitroblue Tetrazolium; BCIP means 5′Bromo, 4′ chloro, 3-indolyl phosphate; ELISA means Enzyme Linked Immunosorbent Assay; IMDM means Iscove's Modified Delbecco's Medium; TMB means tetramethylbenzidine; MTT means (3-(4,5-Dimethylthiazol-2-yl)-2,5-diphenyltetrazolium bromide); SDS means Sodium Dodecyl Sulphate; RPMI means Rosewell Park Memory Institute; and cAMP means 3′-5′-cyclic adenosine monophosphate.

With aim to provide solution for passive immunization of anthrax, the inventors have found that if N-terminal domain (domain-I) of lethal factor (LF) having sequences similar to sequences of N-terminal domain (domain-I) of edema factor (EF) is immunized in a mouse, it surprisingly and unexpectedly results in a monoclonal antibody which has been surprisingly and unexpectedly found to have capability of binding with (or recognizing or cross reacting with) not only lethal factor (LF), but also with edema factor (EF). Additionally, the monoclonal antibody produced has been surprisingly and unexpectedly found to have capability of neutralizing not only the lethal toxin (LT), but also the edema toxin (ET) of B. anthracis, meaning thereby a single monoclonal antibody produced has been surprisingly and unexpectedly found to be capable of curing the patients already infected with anthrax.

The inventors have found that if whole molecule of lethal factor (LF) or even any of its other domains—domain-II, domain-III and domain-IV are immunized in mouse these do not result in monoclonal antibody having above-described characteristics, but results in monoclonal antibody having capability to bind with LF and to neutralize the LT toxin only.

Accordingly, the present invention relates to a monoclonal antibody having capability of binding (or recognizing or cross reacting) with not only lethal factor (LF), but also with edema factor (EF), and capability of neutralizing not only the lethal toxin (LT), but also the edema toxin (ET) of B. anthracis, meaning thereby single monoclonal antibody of present invention is capable of curing the patients already infected with anthrax.

Accordingly, the present invention provides a therapeutic monoclonal antibody which has been found to be capable of treating patients already infected with anthrax and who could not be diagnosed at early stages of infection or were not vaccinated prior to the infection.

In accordance with preferred embodiment of present invention, the monoclonal antibody is of mouse origin, more preferably of BALB/cJ mouse origin.

Accordingly, in one embodiment, the present invention relates to a monoclonal antibody being capable of binding (or recognizing or cross reacting) with lethal factor (LF) and with edema factor (EF), and being capable of neutralizing the lethal toxin (LT) and the edema toxin (ET) of B. anthracis for curing the animals and patients already infected with anthrax or who could not be diagnosed at early stages of infection or were not vaccinated prior to the infection, wherein the monoclonal antibody is of mouse origin.

In accordance with present invention, the mouse origin monoclonal antibody is of BALB/c mouse origin.

In accordance with one of the preferred embodiments of the present invention, the mouse origin monoclonal antibody is of BALB/cJ mouse origin

In accordance with present invention, the monoclonal antibody is obtained by immunizing a mouse with N-terminal domain (domain-I) of lethal factor (LF) having sequences similar to sequences of N-terminal domain (domain-I) of edema factor (EF).

In accordance with present invention, the mouse is preferably immunized with recombinant N-terminal domain of the Lethal factor (rLFn).

In accordance with present invention, the recombinant N-terminal domain of the Lethal factor (rLFn) is 1 to 260 long amino acid fragment of a 809 long amino acid Lethal Factor protein having GenBank Identification Number 301068204 in ‘Protein’ sequence database of GenBank.

In accordance with present invention, the monoclonal antibody is secreted by a hybridoma.

Accordingly, in one embodiment of the present invention, there is also provided a process to prepare the hybridoma secreting the monoclonal antibody of present invention having above-described capabilities.

In accordance with present invention, the process for preparation of hybridoma secreting the monoclonal antibody of present invention comprises steps of immunizing BALB/c mice with the recombinant N-terminal domain of the Lethal factor (rLFn), after the accomplishment of high titer serum response to LFn, the mice were sacrificed and the extracted splenocytes were fused with mouse myeloma cells to obtain said hybridoma capable of secreting the monoclonal antibody of present invention, and the monoclonal antibody of present invention is isolated therefrom.

Accordingly, in one embodiment, the present invention also relates to a process for preparation of monoclonal antibody being capable of binding with (or recognizing or cross reacting with) lethal factor (LF) and also with edema factor (EF), and being capable of neutralizing the lethal toxin (LT) and also the edema toxin (ET) of B. anthracis, comprising steps of:
a) immunizing BALB/c mice with the N-terminal domain (domain-I) of Lethal Factor (LF),
b) after accomplishment of high titer serum response to LF, the mice are sacrificed and the spleen cells are extracted therefrom;
c) extracted spleen cells are fused with mouse myeloma cells to produce hybridoma being capable of secreting the monoclonal antibody of present invention;
d) isolating the monoclonal antibody from hybridoma of step—c).

In accordance with present invention, the N-terminal domain (domain-I) of Lethal Factor (LF) is recombinant N-terminal domain of the Lethal Factor (rLFn).

In accordance with present invention, the recombinant N-terminal domain of the Lethal factor (rLFn) is 1 to 260 long amino acid fragment of a 809 long amino acid Lethal Factor protein having GenBank Identification Number 301068204 in ‘Protein’ sequence database of GenBank.

In accordance with one of the preferred embodiments of present invention, the cells of spleen from LFn-immunized mice are fused to SP2/O myeloma cells.

In accordance with further preferred embodiment of present invention, the cells of spleen from LFn-immunized mice are fused to SP2/O myeloma cells at a ratio of about 4:1.

In accordance with one of the preferred embodiments of present invention, the hybridoma was grown in Iscove's Modified Dulbecco's Medium supplemented with Fetal Bovine Serum

In accordance with one of the preferred embodiments of present invention, the BALB/c mouse is BALB/cJ mouse.

In accordance with one of the preferred embodiments of present invention, the said monoclonal antibody is obtained after immunization of BALB/cJ mouse with N-terminal domain (domain-I) of lethal factor (LF) having sequences similar to sequences of N-terminal domain (domain-I) of edema factor (EF) from the cells of spleen of BALB/cJ mouse.

In accordance with one of the preferred embodiments of present invention, the said monoclonal antibody is obtained after immunization of BALB/cJ mouse with N-terminal domain (domain-I) of lethal factor (LF) having sequences similar to sequences of N-terminal domain (domain-I) of edema factor (EF) from the B cells of spleen of BALB/cJ mouse.

The monoclonal antibody provided herein is a bispecific monoclonal antibody which has also been found to be capable of combining with pharmaceutically acceptable carrier to result in a protective pharmaceutical preparation.

Accordingly, in one embodiment, the present invention also relates to a pharmaceutical preparation comprising a pharmaceutically acceptable carrier and prophylactically effective amount of mAb of present invention which is suitable for prophylaxis against anthrax. The carriers as used herein are nontoxic material that do not interfere with the effectiveness of the biological activity of active ingredients and are selected from group comprising diluents, fillers, salts, buffers, stabilizers, solubilizers, and other materials.

Further, the bispecific monoclonal antibody of present invention has been found to be capable of being combined with antibiotic regimen which have been used for the treatment or amelioration of anthrax disease.

Accordingly, in one embodiment, the present invention also relates to a pharmaceutical preparation comprising antibiotic regimen and prophylactically effective amount of mAb of present invention which is suitable to generate a potent anti-anthrax strategy. The antibiotic regimen, as used herein are regulated course of antibiotic designed to kill B. anthracis and include fluoroquinolones.

Further, the bispecific monoclonal antibody of present invention has been found to be capable of being bound to different carriers and being used in in-vitro studies to detect the presence of one or both of LF and EF which has helped in designing of anthrax diagnostic tool.

It has been observed that monoclonal antibody of present invention is suitable for in-vitro and in-vivo use to monitor the course of anthrax. Therefore, for example, by measuring the concentration of anthrax LF and/or EF present in the body or in various body fluids, it would be possible to determine whether a particular therapeutic regimen aimed at ameliorating anthrax is effective.

Accordingly, in one embodiment, the present invention also relates to anthrax diagnostic tool comprising mAb of present invention bound to different labels and capable of being used in-vitro to detect the presence of LF and/or EF. The labels as used herein, are selected from a group comprising enzymes, paramagnetic or radioisotopes, fluorescent compounds, colloidal metals, chemiluminescent compounds and bioluminescent compounds and other labels known in art.

In accordance with one of the preferred embodiment, the present invention also relates to anthrax diagnostic tool comprising mAb of present invention bound to different carriers and capable of being used in-vitro to detect the presence of LF and/or EF. The nature of the carrier may be soluble or insoluble, and may be selected from the group comprising glass, polystyrene, polypropylene, polyethylene, dextran, nylon, amylase, natural and modified cellulose, polyacrylamide, agarose, and magnetite.

Further, the bispecific monoclonal antibody of present invention has also been found to be capable of being labeled with a paramagnetic or a radioisotope, which have been found suitable for in-vivo diagnostic imaging to assess the progress of disease in anthrax patients.

Accordingly, in yet another embodiment, the present invention also relates to an in-vivo diagnostic imaging wherein mAb of present invention is labeled with a paramagnetic isotope or a radioisotope depending on the detection instrument available which have been found to be capable of being used for in-vivo diagnosis of the presence of LF and/or EF. The paramagnetic isotope as used herein are isotopes suitable for magnetic resonance imaging (MRI) or electron spin resonance (ESR) and are selected from group comprising ¹⁵⁷Gd, ⁵⁵Mn, ¹⁶²Dy, ⁵²Cr and ⁵⁶Fe. The radioisotope as used herein are isotopes that have half-life suitable for detection at the time of maximum uptake but short enough such that deleterious radiation with respect to the host is acceptable and are selected from the group comprising ¹¹¹In, ⁹⁷Ru, ⁶⁷Ga₅, ⁷²As, ⁸⁹Zr and ²⁰¹Tl.

Further, the bispecific monoclonal antibody of present invention has also been found capable of being genetically modified, which have been found to have enhanced binding capability for LF and EF. The genetically modified monoclonal antibody of present invention have been found suitable to form truncated versions that retain their biological function of binding (or recognizing or cross reacting) not only with lethal factor (LF), but also with edema factor (EF), and capability of neutralizing not only the lethal toxin (LT), but also the edema toxin (ET) of B. anthracis, meaning thereby single truncated version of monoclonal antibody of present invention is capable of curing the patients already infected with anthrax. The genetically modified bispecific monoclonal antibody of present invention has also been found suitable for human use.

Accordingly, in one embodiment, the present invention also relates to genetically modified mAb of present invention and usage thereof for making the monoclonal antibody of the present invention more suitable for human use, which is preferably humanized version of whole molecule, and native or humanized antigen binding active fragments (truncated versions) selected from group comprising F(ab′)₂, Fab, Fv and Fd. Or native/humanized F(ab′)₂, Fab, Fv and Fd, wherein each combined with the Fc fragment of any vertebrate origin so as to increase the half-life of these molecules in the body of the patients and thus making their protective efficacy better.

It may be noted that Fab fragment consists of a covalently bound antibody light chain and a portion of the antibody heavy chain denoted as Fd. Fab fragment containing the heavy chain hinge are referred to as Fab′; F(ab′)₂ consists of two Fab′ fragments linked by interchain disulfide bonds. The Fd fragment is the major determinant of antibody specificity and retain epitope binding ability. Fv is the variable domain of the antibody. The portion of an antibody that has a propensity to self-associate and to crystallize into a lattice is called Fc (Fragment, crytallizable) region.

Further, the bispecific monoclonal antibody of present invention has also been found to be capable of protecting mice from lethal anthrax challenge when it is pre-administered or administered after twenty four hours of challenge to mice.

In one embodiment, the present invention also relates to a method for prophylaxis against anthrax disease wherein prophylactically effective amount of mAb of present invention is administered to an animal or human being.

Accordingly, in accordance with present invention, the mAb or genetically modified mAb of present invention may be injected to any animal or human being. The animal, where presently provided mAb or genetically modified mAb may be injected include fowl selected from a group comprising ducks, turkeys, chicken, or a vertebrate, selected from a group comprising fish, amphibian, reptile, bird, or a mammal selected from a group comprising mouse, dog, cat, goat, sheep, horse, pig, cow, human being.

It has been observed that anti-anthrax therapy when performed by employing single therapeutic monoclonal antibody of present invention neutralizes both toxins—LT and ET of B. anthracis, meaning thereby the monoclonal antibody of present invention is expected to be suitable for treating the patients already infected with anthrax by use of said single monoclonal antibody, and hence, probabilities of allergic reactions in human beings is at least avoided.

It may be noted that terms, “prophylaxis” and “therapy” as used herein in conjunction with monoclonal antibodies of present invention denote both prophylactic as well as therapeutic administration and passive immunization with substantially purified polypeptide products. Therefore, the monoclonal antibodies can be administered to high-risk subjects in order to lessen the likelihood and/or severity of anthrax disease or administered to subjects already evidencing active anthrax infection.

The above-described characteristics of bispecific monoclonal antibody of present invention have also been established by the inventors.

In the following description, the term mAb means bispecific monoclonal antibody of present invention, which the inventors have identified as H10 monoclonal antibody.

### 1. Recognition of EF and LF by mAb:

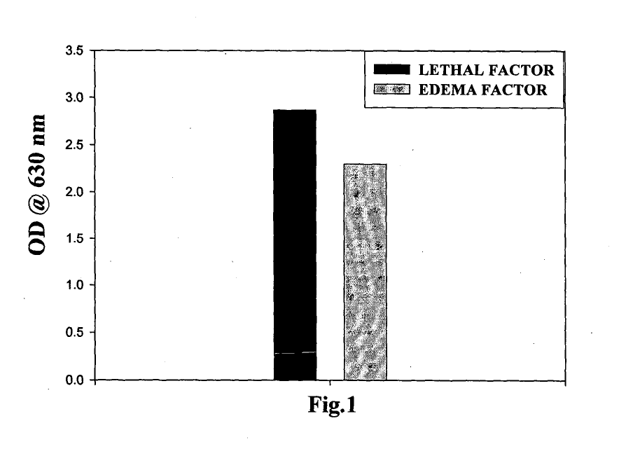

The capability of mAb of present invention to recognize Lethal Factor (LF) and Edema Factor (EF) has been studied after immobilization in 96-well ELISA plates, wherein mAb was used as primary antibody to probe EF and LF, and it was found that mAb recognizes EF as well as LF as also illustrated in accompanying FIG. 1, wherein optical density (OD) of the value of more than about 2.0 is indicative of high binding capability of mAb.

It may be noted that in accordance with present invention, the ELISA may be performed by any known method. The preferred method to perform ELISA has been described in following examples.

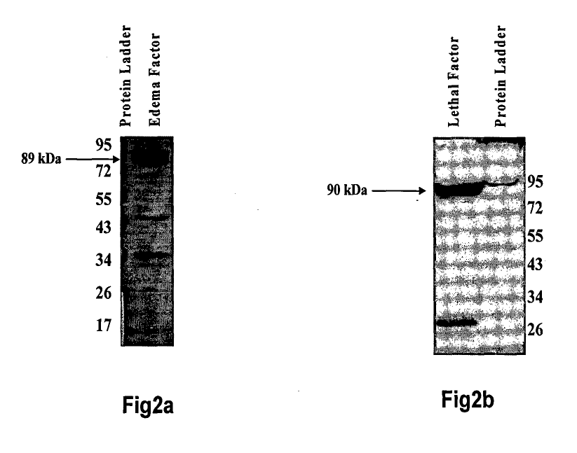

Additionally, capability of mAb of present invention to recognize LF and EF was also studied by Western Blotting, wherein E. coli expression lysates of LF and EF were run by SDS PAGE and transferred to nitrocellulose membrane, which were probed with hybridoma supernatant as primary antibody followed by a suitable detection system including a standard secondary incubation and development of blot, and it was found that mAb recognized EF as visualized by the band at 89 kDa [FIG. 2a], on nitrocellulose membrane, signifying the molecular weight of Edema Factor, and LF as visualized by the band at 90 kDa [FIG. 2b], on nitrocellulose membrane, signifying the molecular weight of Lethal Factor, both confirmed by employing a protein molecular weight ladder.

### 2. In Vitro Neutralization Assay of Lethal Toxin (LT) by mAb:

The capability of mAb of present invention to neutralize cytolytic activity of Lethal Toxin (LT) was evaluated by mixing LT with different dilutions of hybridoma supernatant containing mAb of present invention.

In accordance with one of the preferred embodiments of present invention, the in vitro neutralization assay of LT by mAb was studied by seeding the murine macrophage-like cell line, J774A.1 at a density of about 2×10⁴ cells per well of tissue culture-treated 96 well plate (costar, N.Y.) followed by treating the cells with saturating concentration of LT only or of LT premixed with different dilutions of mAb or only IMDM for the purpose to check survival of the cells in the absence of the LT. It is a control that is put to check if the media itself is not causing any deletrious effect. It was found that the cells incubated with LT only showed 0% survival, the cells treated with LT pre-mixed with the lowest dilution of mAb showed maximum survival and neutralization of LT decreased as the mAb dilution increased. This study confirms that cytolytic effect (or activity) of LT could be neutralized by mAb of present invention, and the cell line could be protected from undergoing Programmed Cell Death.

### 3. In Vitro Neutralization Assay of Edema Toxin (ET) by mAb:

The capability of mAb of present invention to neutralize adenylate cyclase activity of Edema Toxin (ET) was evaluated by mixing ET with different dilutions of hybridoma supernatant containing mAb of present invention.

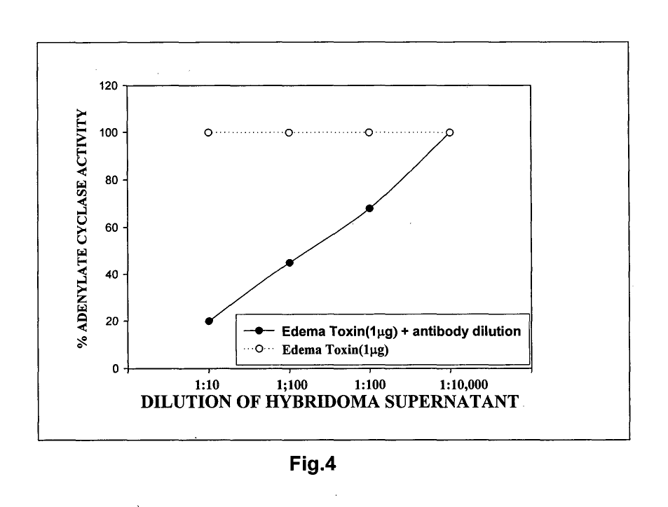

In accordance with one of the preferred embodiments of present invention, the in vitro neutralization assay of ET by mAb was evaluated by assaying the adenylate cyclase activity of saturating concentrations of ET on CHO.K1 cell line which was seeded in wells of tissue culture-treated 96 well plate (costar, N.Y.) and treated with only ET or ET pre-mixed with mAb or only with RPMI media for the purpose to check the cAMP levels in normal physiological condition, in the absence of ET. It is a control which is absolutely essential to check if the media is not causing the observed stress. If the stress seen in these wells is same as in the toxin treated wells then the experiment is null and void. It was found that the wells in which cells were incubated with ET pre-mixed with the lowest dilution of H10 mAB-containing hybridoma supernatant there was maximum neutralization of ET, and the neutralization of ET declined as the mAb was diluted. This indicated that abundance of mAb of present invention was capable of neutralizing adenylate cyclase activity of ET [FIG. 4] and protecting the cells from physiological stress of edema toxins.

### 4. Animal Protection by mAb:

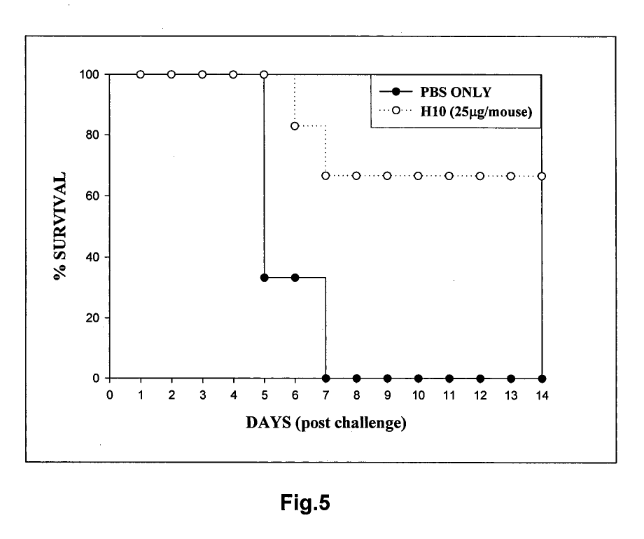

To access capability of mAb of present invention to protect animals on its pre-administration, BALB/c mice were injected with mAb of present invention. After about twenty four hours of this priming, two groups of said mice were challenged with vegetative bacilli of B. anthracis. Survival percentage of mice treated with mAb of present invention which was administered intraperitoneally twenty four hours prior to anthrax inoculation was found to be about 67% as against zero survival percentage of the group administered only with PBS confirming that mAb of present invention is also capable of protecting the animals on its pre-administration as illustrated in accompanying FIG. 5.

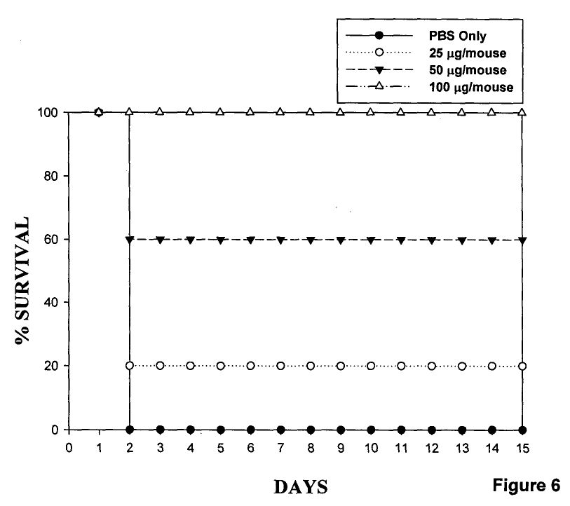

To access the capability of mAb of present invention to protect animals from in-vivo lethal toxin (LT) challenge three groups of six-eight weeks old female BALB/c mice were intraperitoneally immunized with single dose of 25 μg or 50 μg or 100 μg of mAb of present invention. The control group was given only PBS. After twenty four hours mice were challenged with Lethal Toxin (2× LD₅₀). Mice passively immunized with purified monoclonal antibody were protected from lethal toxin. Mice were observed every twenty four hours and surviving mice were sacrificed after fifteenth day. All the mice of control group died after toxin challenge. However, the mAb of present invention gave 20% and 60% protection to groups of mice which were injected, respectively, with 25 μg and 50 μg of mAb of present invention as can be observed in accompanying FIG. 6. However, the mAb of present invention, surprisingly and unexpectedly, gave 100% protection to groups of mice which were injected with 100 μg of mAb of present invention as can be observed in accompanying FIG. 6. This confirms that mAb of present invention is also capable of protecting the animals from lethal toxin.

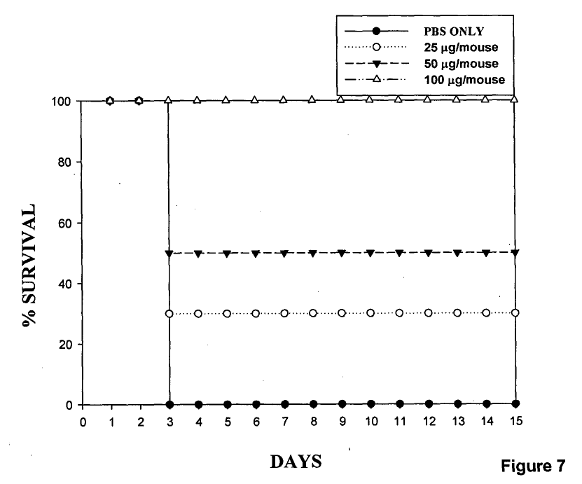

To access the capability of mAb of present invention to protect animals from in-vivo edema toxin (ET) challenge three groups of six-eight weeks old female BALB/c mice were intraperitoneally immunized with single dose of 25 μg or 50 μg or 100 μg of mAb of present invention. The control group was given only PBS. After twenty four hours mice were challenged with Edema Toxin (2× ED₅₀). Mice passively immunized with purified monoclonal antibody were protected from edema toxin. Mice were observed every twenty four hours and surviving mice were sacrificed after fifteenth day. All the mice of control group died after toxin challenge. However, the mAb of present invention gave 30% and 50% protection to groups of mice which were injected, respectively, with 25 μg and 50 μg of mAb of present invention as can be observed in accompanying FIG. 7. However, the mAb of present invention, surprisingly and unexpectedly, gave 100% protection to groups of mice which were injected with 100 μg of mAb of present invention as can be observed in accompanying FIG. 7. This confirms that mAb of present invention is also capable of protecting the animals from edema toxin.

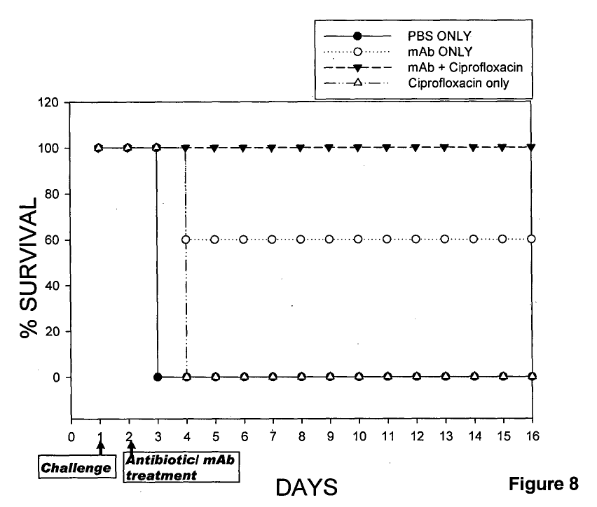

To access the capability of mAb of present invention to protect animals from in-vivo anthrax challenge four groups of six-eight weeks old female BALB/c mice were included in the study. Each group was challenged with 3×10⁶ CFU of B. anthracis. After twenty four hours of challenge, each group was intraperitoneally injected with 4 mg/kg of mAb only or 4 mg/kg of mAb and 8 mg/kg of ciprofloxacin or 8 mg/kg of ciprofloxacin only. The control group was given only PBS. Groups injected with ciprofloxacin received 8 mg/kg dose of antibiotic daily at a gap of twenty four hours for fifteen days after challenge. All the mice of control group and mice injected with only ciprofloxacin died after anthrax challenge. However, the mice injected with 4mg/kg of mAb demonstrated protection of 60% of mice in the group while mice injected with mAb, surprisingly and unexpectedly, demonstrated 100% protection when it was combined with 8 mg/kg dose of ciprofloxacin (a fluoroquinolone) as illustrated in accompanying FIG. 8. This confirms that the antibody of present invention is effective as a post exposure prophylactic agent if combined with appropriate antibiotic regimen.

### EXAMPLES

The present invention is now described with the help of following examples, which are not intended to limit its scope.

### Example I

### 1. Recognition of EF and LF by mAb:

The ELISA for confirmation of capability of mAb of present invention to recognize LF and EF may be performed as follows:

The ELISA plate was coated with EF and LF in separate wells at a concentration of 1 μg/well in PBS (pH 7.5) and incubated for 16 h at 4° C. in triplicate. The wells were washed with 0.05% PBS/Tween 20 and blocked with 200 μl of 2% BSA-PBS for 1 h at 37° C. The neat hybridoma supernatant was added to each well in a volume of 100 μl and incubated for one hour. For negative control, FBS supplemented with IMDM was added in an amount of about 100 μl to the wells coated with EF and LF in same volume and for same time in triplicate. The wells were washed with 0.05% PBS/Tween 20 and incubated with 1:10,000 dilution of horseradish peroxidase conjugated sheep anti-mouse IgG for 1 h at 37° C. The color was developed by adding TMB and absorbance (OD) was measured at 630 nm in a microplate ELISA reader (Bio-Rad) and was found to of the value of more than about 2.0 which indicates that mAb has high binding capability.

The Western Blotting for additional confirmation of capability of mAb of present invention to recognize LF and EF may be performed as follows:

The E. coli expression lysates of LF and EF were run by SDS PAGE for overnight and transferred to nitrocellulose membrane, which was probed with hybridoma supernatant containing H10 mAb for about one hour followed by washings with PBST (PBS and 0.1% Tween20), it was subjected to secondary antibody incubation by adding anti-mouse IgG-Alkaline Phoshatase linked antibody at about 1:10,000 dilution for about one hour, which on washings with PBST (PBS and 0.1% Tween20) and development with NBT/BCIP in alkaline Phosphate buffer resulted in development of the blot, wherein band at 89 kDa [FIG. 2a], on nitrocellulose membrane, signifying the molecular weight of Edema Factor indicates that mAb of present invention recognizes EF, and band at 90 kDa [FIG. 2b], on nitrocellulose membrane, signifying the molecular weight of Lethal Factor, indicates that mAb of present invention also recognizes LF.

### 2. In Vitro Neutralization Assay of Lethal Toxin (LT) by mAb:

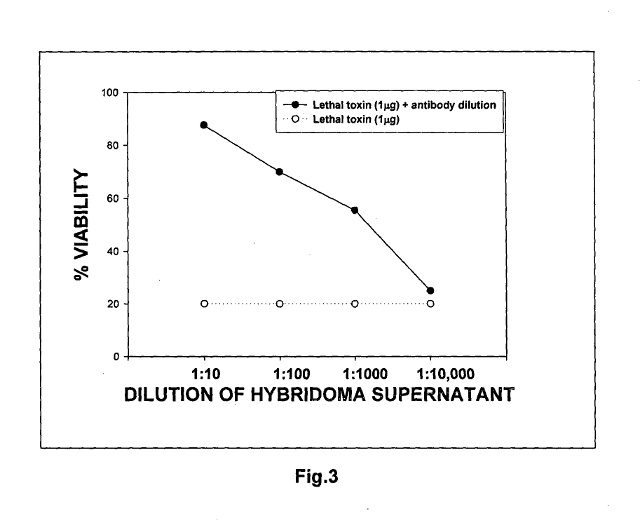

The in vitro neutralization assay of LT by mAb was studied by seeding the murine macrophage-like cell line, J774A.1 at a density of about 2×10⁴ cells per well of tissue culture-treated 96 well plate (costar, N.Y.) followed by treating the cells with saturating concentration of about 1 μg/ml of LT or about 1 μg/ml of LT premixed with different dilutions of mAb (H10 hybridoma supernatant) or only IMDM for about three hours followed by addition of about 0.5 mg/ml of MTT (3-(4,5-Dimethylthiazol-2-yl)-2,5-diphenyltetrazolium bromide) for about forty five minutes. The reaction mix was removed and oxidized MTT was solubilized by the addition of about 100 μl of solubilizaton buffer (25 mM HCl, 0.05% SDS in 90% isopropanol). The OD of only IMDM treated-cells was taken as the OD of 100% viable cells and viability of other wells was calculated with this reference. The OD measured at 570 nm at Tecan (sunrise) ELISA reader [FIG. 3] indicates that the cells treated with LT premixed with the lowest dilution of mAb showed maximum survival percentage and as the dilution of the hybridoma supernatant that contained H10 mAb increased, the neutralization of LT decreased.

### 3. In Vitro Neutralization Assay of Edema Toxin (ET) by mAb:

The in vitro neutralization assay of ET by mAb was studied by assaying the adenylate cyclase activity of saturating concentrations of about 1 μg/ml of ET on CHO.K1 cell line which was seeded for 80% confluence in wells of tissue culture-treated 96 well plate (costar, N.Y.) and treated only with about 1 μg/ml of ET or with about 1 μg/ml of ET pre-mixed with mAb (H10 hybridoma supernatant) or only with RPMI media for about three hours. Total cAMP levels were measured using cyclic AMP competitive ELISA kit from Thermo Scientific, Pierce protein research product, according to manufacturer's protocol.

It was found that when cell line is incubated with ET in presence of mAb of present invention, the cyclic AMP production decreased indicating that abundance of mAb of present invention was capable of neutralizing adenylate cyclase activity of ET.

### 4. Animal Protection by mAb:

The ability of mAb of present invention to protect animals on its pre-administration was tested on BALB/c mice which were pre-injected with mAb of present invention. After about twenty four hours of this priming, two groups of said mice were challenged with vegetative bacilli of B. anthracis. Survival percentage of mice treated with mAb of present invention which was pre-administered intraperitoneally twenty four hours prior to anthrax inoculation was found to be about 67% as against zero survival percentage of the group administered only with PBS confirming that mAb of present invention is also capable of protecting the animals on its pre-administration [FIG. 5].

The ability of mAb of present invention to protect animals from in-vivo lethal toxin challenge was tested. Three groups of six-eight weeks old female BALB/c mice, ten in number, were intraperitoneally immunized with single dose of 25 g or 50 μg or 100 μg of mAb of present invention. The control group was given only PBS. After twenty four hours mice were challenged with Lethal Toxin (2× LD₅₀). Mice passively immunized with purified monoclonal antibody were protected from lethal toxin. Mice were observed every twenty four hours and surviving mice were sacrificed after fifteenth day. All the mice of control group died after toxin challenge. However, the mice injected with mAb of present invention, surprisingly and unexpectedly, demonstrated 20%, 60%, and even 100% protection to groups of mice when injected, respectively, with 25 μg, 50 μg and 100 μg of mAb of the present invention [FIG. 6]. This study confirms that mAb of present invention is also capable of protecting the animals from lethal toxin.

The ability of mAb of present invention to protect animals from in-vivo edema toxin challenge was tested. Three groups of six-eight weeks old female BALB/c mice, ten in number, were intraperitoneally immunized with single dose of 25 μg or 50 μg or 100 μg of mAb of present invention. The control group was given only PBS. After twenty four hours mice were challenged with Edema Toxin (2× ED₅₀). Mice passively immunized with purified monoclonal antibody were protected from edema toxin. Mice were observed every twenty four hours and surviving mice were sacrificed after fifteenth day. All the mice of control group died after toxin challenge. However, the mice injected with mAb of present invention, surprisingly and unexpectedly, demonstrated 30%, 50%, and even 100% protection to groups of mice when injected, respectively, with 25 μg, 50 μg and 100 μg of mAb of the present invention [FIG. 7]. This study confirms that mAb of present invention is also capable of protecting the animals from edema toxin.

The ability of mAb of present invention to protect animals from in-vivo anthrax challenge was tested. Four groups of six-eight weeks old female BALB/c mice, ten in number, were included in the study. Each group was challenged with 3×10⁶ CFU of B. anthracis. After twenty four hours after challenge, each group was intraperitoneally injected with 4 mg/kg of mAb only or 4 mg/kg of mAb and 8 mg/kg of ciprofloxacin or 8 mg/kg of ciprofloxacin only. The control group was given only PBS. Groups injected with ciprofloxacin received 8 mg/kg dose of antibiotic daily at a gap of twenty four hours. All the mice of control group and mice injected with ciprofloxacin only died after anthrax challenge. However, the mice injected with 4mg/kg of mAb of present invention demonstrated protection of 60% in the group while it, surprisingly and unexpectedly, gave 100% protection to the mice when these were injected with combination of 4 mg/kg of mAb of present invention and 8 mg/kg of ciprofloxacin [FIG. 8]. This study confirms that the antibody of present invention is effective as a post exposure prophylactic agent if combined with appropriate antibiotic dose.

Accordingly, it is understood that presently provided bispecific monoclonal antibody provides a solution for passive immunization against anthrax. As present invention provides a monoclonal antibody which alone has been found to be capable of binding with (or recognizing) not only lethal factor (LF), but also with edema factor (EF), and capable of neutralizing not only lethal toxin (LT), but also edema toxin (ET) of B. anthraces, there is provided a solution for passive immunization against anthrax, wherein probabilities of allergic reactions is also avoided.

1. A monoclonal antibody for curing the patients or animals already infected with anthrax or who could not be diagnosed at early stages of infection or were not vaccinated prior to the infection by passive immunization against anthrax, wherein said monoclonal antibody binds (or recognizes or cross reacts) with lethal factor (LF) and with edema factor (EF), and neutralizes the lethal toxin (LT) and the edema toxin (ET) of B. anthracia, wherein further the monoclonal antibody is of mouse origin.

2. The monoclonal antibody as claimed in claim 1, wherein it is of BALB/c mouse origin or BALB/cJ mouse origin.

3. (canceled)

4. The monoclonal antibody as claimed in claim 1, wherein it is obtained by immunizing a mouse with N-terminal domain (domain-I) of lethal factor (LF) having sequences similar to sequences of N-terminal domain (domain-I) of edema factor (EF).

5. The monoclonal antibody as claimed in claim 1, wherein mouse is immunized with recombinant N-terminal domain of the Lethal Factor (rLFn), and said recombinant N-terminal domain of the Lethal factor (rLFn) is 1 to 260 long amino acid fragment of a 809 long amino acid Lethal Factor protein having GenBank Identification Number 301068204 in ‘Protein’ sequence database of GenBank.

6. (canceled)

7. A process for preparation of monoclonal antibody as claimed in claim 1, wherein said monoclonal antibody binds (or recognizes or cross reacts) with lethal factor (LF) and with edema factor (EF), and neutralizes the lethal toxin (LT) and the edema toxin (ET) of B. anthracis, comprising steps of:
a) immunizing BALB/c mouse with N-terminal domain (domain-I) of Lethal Factor (LF),
b) after accomplishment of titer serum response to LF, the mice are sacrificed and the spleen cells are extracted therefrom;
c) extracted spleen cells are fused with mouse myeloma cells to produce hybridoma being capable of secreting the monoclonal antibody;
d) isolating the monoclonal antibody hybridoma of step—c).

8. The process as claimed in claim 7, wherein N-terminal domain is recombinant N-terminal domain of the Lethal Factor (rLFn), and said recombinant N-terminal domain of the Lethal factor (rLFn) is 1 to 260 long amino acid fragment of a 809 long amino acid Lethal Factor protein having GenBank Identification Number 301068204 in ‘Protein’ sequence database of GenBank.

9. (canceled)

10. The process as claimed in claim 7, wherein cells of spleen from LFn-immunized mice are fused to SP2/0 myeloma cells.

11. The process as claimed in claim 7, wherein cells of spleen from LFn-immunized mice are fused to SP2/0 myeloma cells at a ratio of about 4:1.

12. The process as claimed in claim 7, wherein hybridoma is grown in Iscove's Modified Dulbecco's Medium supplemented with Fetal Bovine Serum.

13. The process as claimed in claim 7, wherein BALB/c mouse is BALB/cJ mouse.

14. The process as claimed in claim 7, wherein said monoclonal antibody is obtained from spleen cells of BALB/cJ mouse after immunization of BALB/cJ mouse with N-terminal domain (domain-I) of lethal factor (LF), wherein said N-terminal domain (domain-I) of lethal factor (LF) has sequences similar to sequences of N-terminal domain (domain-I) of edema factor (EF).

15. The process as claimed in claim 7, wherein said spleen cells are B cells of spleen of BALB/cJ mouse.

16. (canceled)

17. (canceled)

18. The pharmaceutical preparation as claimed in claim 35, wherein monoclonal antibody is of mouse origin and binds (or recognizes or cross reacts) with lethal factor (LF) and with edema factor (EF), and neutralizes the lethal toxin (LT) and the edema toxin (ET) of B. anthracis, combined with antibiotic regimen which is suitable for the treatment or amelioration of anthrax disease.

19. The A pharmaceutical preparation as claimed in claim 18, wherein antibiotic regimen includes fluoroquinolones.

20. Anthrax diagnostic tool which comprises monoclonal antibody as claimed in claim 1.

21. Anthrax diagnostic tool as claimed in claim 20, wherein monoclonal antibody is bound to labels selected from enzymes, paramagnetic or radioisotopes, fluorescent compounds, colloidal metals, chemiluminescent compounds and bioluminescent compounds.

22. Anthrax diagnostic tool as claimed in claim 20, wherein monoclonal antibody is bound to carrier selected from glass, polystyrene, polypropylene, polyethylene, dextran, nylon, amylase, natural and modified cellulose, polyacrylamide, agarose, and magnetite.

23. Anthrax diagnostic tool as claimed in claim 21, wherein monoclonal antibody is bound to paramagnetic or a radioisotope for in-vivo diagnostic imaging to assess the progress of disease in anthrax patients; wherein said paramagnetic isotope is suitable for magnetic resonance imaging (MRI) or electron spin resonance (ESR) and is selected from ¹⁵⁷Gd, ⁵⁵Mn, ¹⁶²Dy, ⁵²Cr and ⁵⁶Fe; or wherein said radioisotope has half-life suitable for detection at the time of maximum uptake and is selected from ¹¹¹In, ⁹⁷Ru, ⁶⁷Ga₅, ⁷²As, ⁸⁹Zr and ²⁰¹Tl.

24. (canceled)

25. (canceled)

26. (canceled)

27. (canceled)

28. A monoclonal antibody prepared by the process as claimed in claim 5, wherein monoclonal antibody is bispecific monoclonal antibody.

29. A monoclonal antibody as prepared by the process as claimed in claim 5, wherein monoclonal antibody is genetically modified.

30. The monoclonal antibody as claimed in claim 29, wherein genetically modified monoclonal antibody is humanized version of whole molecule, and native or humanized antigen binding active fragments (truncated versions) selected from F(ab′)2, Fab, Fv and Fd or native/humanized F(ab′)₂, Fab, Fv and Fd, wherein each combined with the Fc fragment of any vertebrate origin.

31. A method for treatment or prophylaxis against anthrax disease which counterpoises administering to patient in need thereof an effective amount of monoclonal antibody as claimed in in claim 1.

32. The method as claimed in claim 31 wherein the subject is an animal including fowl selected from ducks, turkeys, chicken, or a vertebrate selected from fish, amphibian, reptile, bird, or a mammal selected from mouse, dog, cat, goat, sheep, horse, pig, cow, human being.

33. (canceled)

34. (canceled)

35. A pharmaceutical preparation which comprises prophylactically effective amount suitable for prophylaxis against anthrax of monoclonal antibody as claimed in claim 1, preferably prepared by the process as claimed in claim 5 and at least one pharmaceutically acceptable carrier selected from diluents, fillers, salts, buffers, stabilizers and/or solubilizers.

36. A pharmaceutical preparation as claimed in claim 35, wherein said monoclonal antibody is obtained from at least one mouse immunized with recombinant N-terminal domain of the Lethal Factor (rLFn), and said recombinant N-terminal domain of the Lethal factor (rLFn) is 1 to 260 long amino acid fragment of a 809 long amino acid Lethal Factor protein having GenBank Identification Number 301068204 in ‘Protein’ sequence database of GenBank.

37. The pharmaceutical composition as claimed in claim 18, wherein said monoclonal antibody is obtained from at least one mouse immunized with recombinant N-terminal domain of the Lethal Factor (rLFn), and said recombinant N-terminal domain of the Lethal factor (rLFn) is 1 to 260 long amino acid fragment of a 809 long amino acid Lethal Factor protein having GenBank Identification Number 301068204 in ‘Protein’ sequence database of GenBank.

38. Anthrax diagnostic tool as claimed in claim 20, wherein said monoclonal antibody is obtained from at least one mouse immunized with recombinant N-terminal domain of the Lethal Factor (rLFn), and said recombinant N-terminal domain of the Lethal factor (rLFn) is 1 to 260 long amino acid fragment of a 809 long amino acid Lethal Factor protein having GenBank Identification Number 301068204 in ‘Protein’ sequence database of GenBank.
